(19) United States
(12) Patent Application Publication
(10) Pub. No.: US 2026/0174372 A1
(43) Pub. Date: Jun. 25, 2026

(54) SYSTEMS AND METHODS FOR MEASURING ECG DATA AND RESPIRATORY DATA FOR A PATIENT

(71) Applicant: GE Precision Healthcare LLC, Wauwatosa, WI (US)

(72) Inventors: Juha Virtanen, Helsinki (FI); Emma Hellman, Espoo (FI); René Coffeng, Helsinki (FI); Terho Pulliainen, Helsinki (FI)

(73) Assignee: GE Precision Healthcare LLC, Wauwatosa, WI (US)

(21) Appl. No.: 19/538,603

(22) Filed: Feb. 12, 2026

Related U.S. Application Data
(63) Continuation of application No. 17/523,315, filed on Nov. 10, 2021.

(51) Int. Cl. A61B 5/28; A61B 5/00; A61B 5/08; A61B 5/273

(52) U.S. Cl. CPC A61B 5/28; A61B 5/08; A61B 5/273; A61B 5/6823

(57) ABSTRACT

A method for monitoring a patient. The method includes connecting a first and a second sets of ECG wires to the patient to receive a first and a second sets of cardiac electrical activity from the patient therewith. The method further includes monitoring the patient based on the first and second sets of cardiac electrical activity when the first and second sets of ECG wires are both connected to the patient. The method further includes disconnecting the second set of ECG wires from the patient without disconnecting the first set of ECG wires from the patient and monitoring the patient based on the first set of cardiac electrical activity when the second set of ECG wires is disconnected from the patient. Optionally connecting the second set of ECG wires allows monitoring in two different configurations without disconnecting the first set of ECG wires from the patient to transition therebetween.

### CROSS REFERENCE TO RELATED APPLICATION

This application is a continuation of U.S. patent application Ser. No. 17/523,315, filed Nov. 10, 2021, which is incorporated herein by reference in its entirety.

### FIELD

The present disclosure generally relates to systems and methods for measuring ECG data and respiratory data for a patient.

### BACKGROUND

Electrocardiograms and the devices that generate these waveforms (also referred to as ECG devices or ECGs) are essential tools in medicine, used frequently within clinical and hospital settings to monitor, diagnose, and treat heart conditions. In particular, electrical activity from a patient's heart is collected via electrodes placed on the skin in specific regions of the body. This electrical activity is also referred to herein as cardiac electrical activity. The cardiac electrical activity is communicated from the electrodes to an electronics device via wires. The electronics device, or another device connected thereto, processes the cardiac electrical activity from the electrodes to measure ECG data (e.g., via comparison between particular electrodes) and to create an ECG waveform. The electronics device or other device connected thereto may also perform other actions based on the cardiac electrical activity, such as generating alarms, creating notifications or displays, and the like in a manner known in the art.

The number of electrodes and wires connected to the patient varies according to the configuration of the ECG device. Common configurations known in the art include: (1) 3-lead, which uses 3 electrodes positioned on the right arm, left arm, and left leg; (2) 5-lead, which uses 5 electrodes positioned on the right arm, right leg, left arm, left leg, and one on the chest; (3) 6-lead, which uses 6 electrodes positioned on the right arm, right leg, left arm, left leg, and two on the chest; and (4) 12-lead, which uses 10 electrodes comprised of four limb leads (right arm, right leg, left arm, left leg) and six chest leads commonly referred to as V1-V6. The six chest leads of a conventional 12-lead ECG are positioned with V1 being at the 4th intercostal space on the right sternum, V2 being at the 4th intercostal space on the left sternum, V3 being midway between V2 and V4, V4 being at the fifth intercostal space at the mid-clavicular line, V5 being at the fifth intercostal space at an anterior axillary line (same horizontal level as V4), and V6 being at the fifth intercostal space at a mid-axillary line (same horizontal level as V4). One example of a 12-lead ECG device in the market is the Carescape One produced by GE Healthcare®.

Some ECG devices are also configured to measure respiratory data representing the breathing characteristics of the patient. The respiratory data is also derived by measuring electrical activity on the skin of the patient (separately referred to as respiratory electrical activity), which in systems and methods presently known in the art is collected from the same electrodes used for collecting the cardiac electrical activity for generating the ECG waveform.

### SUMMARY

This Summary is provided to introduce a selection of concepts that are further described below in the Detailed Description. This Summary is not intended to identify key or essential features of the claimed subject matter, nor is it intended to be used as an aid in limiting the scope of the claimed subject matter.

One example of the present disclosure generally relates to a system for measuring ECG data and respiratory data for a patient. The system includes at least four ECG wires configured to communicate a first set of cardiac electrical activity from the patient. A respiratory wire distinct from the at least four ECG wires is configured to communicate respiratory electrical activity from the patient. An electronics device is electrically coupled to the at least four ECG wires and to the respiratory wire. The electronics device is configured to measure the ECG data based on the first set of cardiac electrical activity from the at least four ECG wires, and to measure the respiratory data based on the respiratory electrical activity from the respiratory wire.

In certain examples, the five ECG wires and the respiratory wire are each configured to be electrically coupled to the patient via electrodes, and a respiratory electrode associated with the respiratory wire is unshared with any of the electrodes associated with the at least four ECG wires.

In certain examples, the electronics device receives an additional electrical activity measured on an abdomen of the patient, and the electronics device measures the respiratory data by comparing the respiratory electrical activity to the additional electrical activity. In further examples, the additional electrical activity is communicated via one of the at least four ECG wires.

In certain examples, the respiratory electrical activity is measured closer to a left armpit of the patient than to a sternum of the patient.

In certain examples, the respiratory wire is a first respiratory wire and the respiratory electrical activity is a first set of respiratory electrical activity measured in a first location on the patient and communicated by the first respiratory wire. A second respiratory wire is also included and is configured to communicate a second set of respiratory electrical activity measured in a second location on the patient, where the electronics device receives additional electrical activity measured on the patient, and where the electronics device measures the respiratory data based on comparison of both the first set of respiratory electrical activity and the second set of respiratory electrical activity to the additional electrical activity.

Certain examples further include electrodes by which the at least four ECG wires and the respiratory wire receive the cardiac electrical activity and the respiratory electrical activity from the patient, respectively, where one of the electrodes is configured to communicate with two separate wires among the respiratory wire and the at least four ECG wires.

In certain examples, the electronics device includes a first electronics device electrically coupled to the at least four ECG wires and the respiratory wire, and a second electronics device electrically coupled to additional ECG wires configured to communicate the cardiac electrical activity measured from the patient, where the ECG data is measured based on the cardiac electrical activity from the at least four ECG wires and also from the additional ECG wires. In further examples, the additional ECG wires are leads V2 through V6 in a conventional 12-lead ECG configuration.

Another example of the present disclosure generally relates to a method for measuring ECG data and respiratory data for a patient. The method includes electrically coupling at least four ECG wires to the patient to communicate a first set of cardiac electrical activity from the patient, where one of the at least four ECG leads is positioned on an abdomen of the patient. The method further includes electrically coupling a respiratory wire to the patient to communicate respiratory electrical activity from the patient, electrically coupling the at least four ECG wires and the respiratory wire to an electronics device. The method further includes configuring the electronics device to measure the ECG data based on the first set of cardiac electrical activity from the at least four ECG wires, and to measure the respiratory data based on the respiratory electrical activity from the respiratory wire.

In certain examples, the one of the five ECG wires positioned on the abdomen of the patient provides an additional electrical activity, where the respiratory wire is positioned closer to a left armpit of the patient than to a sternum of the patient, and where the electronics device measures the respiratory data by comparing the respiratory electrical activity to the additional electrical activity.

In certain examples, the respiratory wire is a first respiratory wire and the respiratory electrical activity is a first set of respiratory electrical activity measured in a first location on the patient and communicated by the first respiratory wire, further comprising electrically coupling a second respiratory wire to the patient to communicate a second set of respiratory electrical activity measured in a second location on the patient, wherein the electronics device receives additional electrical activity measured on the patient, and wherein the electronics device measures the respiratory data based on comparison of both the first set of respiratory electrical activity and the second set of respiratory electrical activity to the additional electrical activity.

Certain examples further include positioning electrodes on the patient by which the at least four ECG wires and the respiratory wire receive the cardiac electrical activity and the respiratory electrical activity therefrom, respectively, where one of the electrodes is configured to communicate with two separate wires among the respiratory wire and the at least four ECG wires.

In certain examples, the electronics device includes a first electronics device electrically coupled to the at least four ECG wires and the respiratory wire, and a second electronics device electrically coupled to additional ECG wires configured to communicate the cardiac electrical activity measured from the patient, where the ECG data is measured based on the cardiac electrical activity from the at least four ECG wires and also from the additional ECG wires. In further examples, the additional ECG wires are leads V2 through V6 in a conventional 12-lead ECG configuration.

Another example according to the present disclosure generally relates to a system for measuring ECG data for a patient. A first electronics device is configured to be electrically coupled to the patient via a first set of ECG wires to receive a first set of cardiac electrical activity from the patient. A second electronics device is configured to be electrically coupled to the patient via a second set of ECG wires to receive a second set of cardiac electrical activity from the patient. A monitoring device is configured to communicate with the first electronics device and the second electronics device, where the monitoring device is configured to measure the ECG data for the patient based on the first set of cardiac electrical activity received from the first electronics device when communication is absent from the second electronics device, and where the monitoring device is configured to measure ECG data for the patient based on both the first set of cardiac electrical activity received from the first electronics device and the second set of cardiac electrical activity received from the second electronics device when communication is present from both the first electronics device and the second electronics device.

In certain examples, the monitoring device is configured to measure ECG data for the patient based on both the first set of cardiac electrical activity and the second set of cardiac electrical activity when at least one of the first set of ECG wires and at least one of the second set of ECG wires are electrically coupled to the patient via a shared electrode positioned thereon. In further examples, the shared electrode provides additional electrical activity for both the first set of ECG wires and the second set of ECG wires, and measuring the ECG data includes comparing each of the first set of cardiac electrical activity and the second set of cardiac electrical activity to the additional electrical activity.

In certain examples, the first electronics device is further configured to be electrically coupled to the patient via a respiratory wire configured to measure respiratory electrical activity for the patient, where the respiratory wire is distinct from the first set of ECG wires, and where the monitoring device is further configured to measure respiratory data for the patient based on the respiratory electrical activity received from the respiratory wire.

Certain examples further relate to methods for using the systems presently disclosed, including electrically coupling the first set of ECG wires to the patient via electrodes, where one of the electrodes is positioned on an abdomen of the patient.

Various other features, objects and advantages of the disclosure will be made apparent from the following description taken together with the drawings.

### BRIEF DESCRIPTION OF THE DRAWINGS

The present disclosure is described with reference to the following drawings.

FIG. 1 is perspective view of a system according to the present disclosure in-use for measuring ECG data for a patient;

FIG. 2 depicts a first configuration of a system according to the present disclosure for measuring ECG data (here providing 5-lead ECG data), also measuring respiratory data;

FIG. 3 is a schematic view of an example control system such as may be incorporated within the systems disclosed herein;

FIG. 4 depicts a second configuration of a system according to the present disclosure for measuring ECG data (here providing 12-lead ECG data);

FIG. 5 depicts a third configuration of a system according to the present disclosure for measuring ECG data (here providing 12-lead ECG data);

FIG. 6 depicts a fourth configuration of a system according to the present disclosure similar to FIG. 4, also measuring respiratory data;

FIG. 7 is a flow chart for a first example of a method for measuring ECG data according to the present disclosure;

FIG. 8 is a flow chart for a second example of a method for measuring ECG data according to the present disclosure;

FIG. 9 is a perspective view of an example of a removable/passthrough connector such as shown in FIG. 2; and

FIG. 10 is a top view of a removable/passthrough connector similar to that of FIG. 9 with a removable portion connected thereto.

### DETAILED DISCLOSURE

It is generally known in the art to use the electrodes measuring ECG data to also make dual vector impedance measurements of respiratory data, for example as described in U.S. Pat. Nos. 7,351,208 and 10,405,765, and U.S. Patent Application Publication No. 2019/0380620. However, the present inventors have recognized that the systems and methods presently known in the art provide inaccurate respiratory data measurements and are generally problematic. For example, low signal amplitude and/or motion artifacts using devices and methods presently known in the art may cause inaccurate respiration rate. A false indication of central apnea is also possible, particularly if the electrodes locations are not optimized to have the strongest signal amplitudes.

In addition, the present inventors have recognized problems when using medical devices and methods presently known in the art, specifically when needing to transition between ECG measuring configurations. For example, in certain cases a patient may be connected to a 5-lead or 6-lead ECG system for a relatively long period of time, such as for extended monitoring (which could range from a hours to several days). In contrast, a 12-lead ECG (which provides much more detailed information regarding the electrical activity of the heart) is typically connected for only short-term collection. For example, a patient arriving at an intensive care unit (ICU) may be checked for possible cardiac issues using a 12-lead ECG, which may require only a few minutes of monitoring or be continued for a few hours. Once the initial monitoring with 12-lead ECG is completed, additional monitoring may be continued using a 5-lead ECG setup. It is also common that a patient already connected to a 5-lead or 6-lead ECG requires a full 12-lead ECG for additional data collection, but will then be subsequently returned back to the 5-lead or 6-lead ECG configuration again. In this scenario, a caregiver must fully remove the entire 5-lead or 6-lead ECG setup from the patient to complete a 12-lead ECG study, then remove the entire 12-lead ECG setup to reapply the 5-lead or 6-lead ECG setup again.

The positioning and removing of electrodes, connecting of wires, and configuration of electronics devices connected thereto is time-consuming for the caregiver, uncomfortable and/or disruptive to the patient, and increases the delay for collecting the additional 12-lead ECG data measurements for the patient (also increasing the time until the patient is restored to the previous configuration). The process also generates additional material cost and waste for multiple rounds of using electrodes, causes additional skin irritation, generates additional wear and tear on the wires, and increase the risk of human error in the placement and connection of the electrodes due to repeated efforts and working under time constraints.

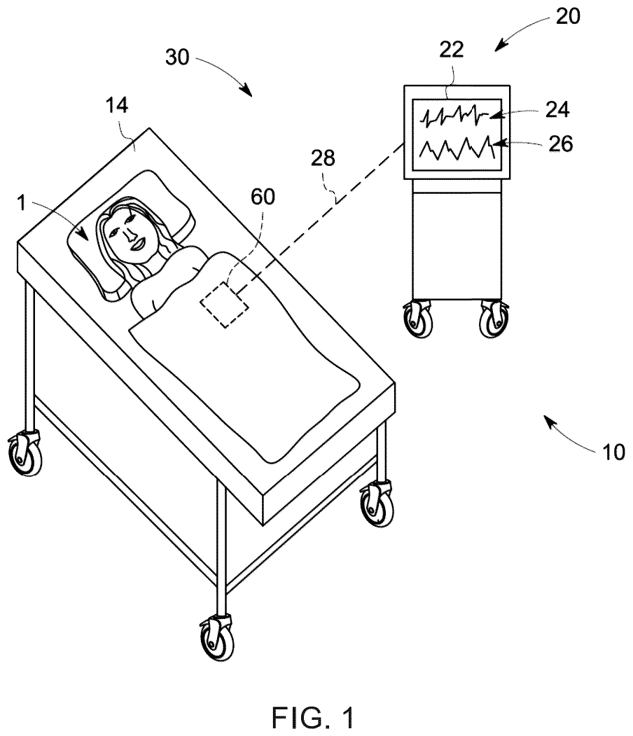

FIG. 1 shows an example configuration of a system 30 for measuring ECG data (and in certain examples, respiratory data) according to the present disclosure. The system 30 includes an electronics device 60 that receives electrical activity from electrodes positioned on a patient 1, as discussed further below. The electronics device can also be referred to as a medical device. The patient 1 may be positioned in a bed 14 as shown, or, due to the flexibility offered by the presently disclosed system 30 (discussed further below), may be free to move, e.g., using a wireless configuration discussed below.

In the example shown, the electronics device 60 communicates via a connection 28 to a separate monitoring device 20, which here has a display device 22 for displaying ECG data 24 and respiratory data 26 collected by the system 30. The connection 28 may be physical, such as wires within a wire harness, and/or wireless, for example using a protocol known in the art (e.g., Bluetooth®, Wi-Fi, or others). The electronics device 60 and/or monitoring device 20 may also communicate with additional devices or systems, such as a central monitoring station or an Electronic Medical Record (EMR) known in the art, for example to display, archive, and/or further process the information collected by the system 30.

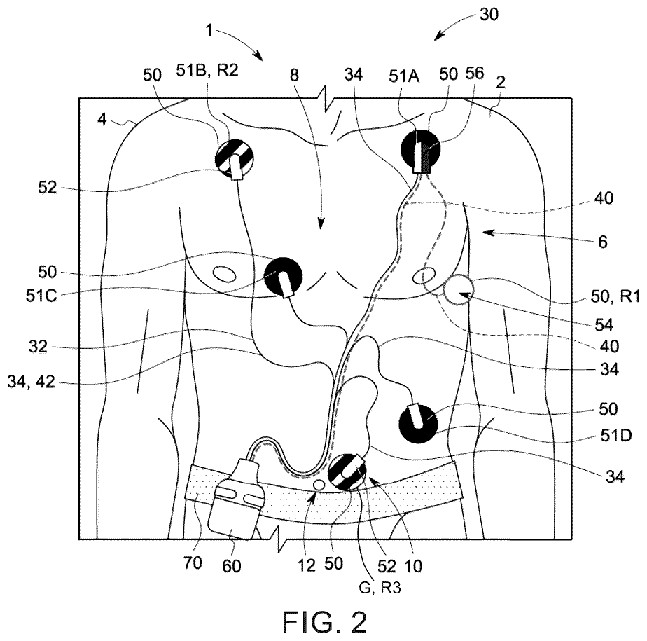

FIG. 2 shows one configuration for measuring both the ECG data and respiratory data for another patient 1. The figure shows the patient's left shoulder 2, right shoulder 4, and abdomen 10. Additional notable landmarks for reference include the left armpit 6, sternum 8, and navel 12. FIG. 2 further shows a number of electrodes 50 coupled to the skin of the patient 1, which may be electrodes presently known in the art unless otherwise stated. The electrodes 50 create electrical signals based on electrical activity present on the surface of the skin, in this case as cardiac electrical activity generated by the beating of the heart, and/or as respiratory electrical activity generated by the patient's breathing. One or more of the electrodes 50 is also used in certain examples as a ground to equalize the potential between the patient and the electronics ground, as is customary in ECG measurement, whereby the electrical activity measured by this electrode is also referred to as additional electrical activity.

For the ease of reference, certain electrodes 50 used exclusively for measuring ECG data are shown in solid black (here also labeled as electrodes 51A, 51C, and 51D). Other electrodes 50 used exclusively for measuring respiratory data are shown in solid white (here also labeled as electrode R1), and those for both ECG data and respiratory (here electrode 51B, R2, and also electrode G, R3) in black and white stripes. However, the actual electrodes 50 used for each purpose (e.g., measuring cardiac, respiratory, and/or additional electrical activity) may be functionally the same, subject to further distinctions described below. It should be recognized that different numbers of electrodes 50 may also be used, for example omitting electrode 51C for a four-lead ECG configuration.

With continued reference to FIG. 2, the electrical signals produced by the electrodes 50 responsive to the electrical activity are then communicated to an electronics device 60 via wires 32 connected therebetween. The wires 32 may be connected to the electronics device 60 and to the electrodes 50 via different methods known in the art, and/or in a manner described further below. It should be recognized that various types of wires 32 known in the art may be used, including shielded and non-shielded, different gauges, and the like. The wires 32 may also be bundled together in a variety of ways, and should thus be broadly considered as individual conductive pathways between points. In certain instances, the wires 32 are separately referred to as ECG wires 34 or respiratory wires 40 to clarify which type of electrical activity is communicated thereby. However, the actual wires used may be the same for any of the types of electrical activity discussed herein (e.g., cardiac, respiratory, and ground). The example shown in FIG. 2 includes five ECG wires 34 (four connecting to electrodes 51A-51D, and one to the ground electrode G), indicating a 5-lead ECG configuration. The electronics device 60 then processes the electronic signals received from the five ECG wires 34 in a manner known in the art to produce the desired ECG data. It should be recognized that the electronics device 60 may also or alternatively communicate these electronic signals to another device (e.g., a monitoring device 20) for processing.

As is discussed further below, ground electrodes G may serve two functions (and thus in certain examples are also labeled as R3). First, the ground electrode G is used for equalizing the potential between human body and the electronics device 60. In the context of measuring ECG data, the additional electrical activity measured by the ground electrode G may not contribute to any of the measurements, whereby the ECG data is instead measured using differential amplifiers all individually referenced to electrode 50 positioned on the right arm (for example). In the context of impedance or respiratory data, the respiratory data may be measured between an electrode positioned to measure respiratory electrical activity (e.g., positioned on the right arm) and another electrode positioned to measure respiratory electrical activities, which is in certain examples the ground electrode G used for measuring the ECG data. Since the ground electrode G also serves the function of measuring respiratory electrical activity, it may also be labeled as electrode R3 (see FIG. 2) to clarify that it measures respiratory electrical activity rather than functioning as a ground in this context. In this manner, the ground electrode G may have two different functions: equalizing potentials at low frequencies, and serving as another pole for the impedance measurement at higher frequencies.

The example FIG. 2 also includes respiratory wires 40, 42 connecting the electrode R2 used for measuring respiratory electrical activity to the electronics device 60. In the specific configuration shown, the connection to the electrode 51A is a removable/passthrough connector 56 specifically developed by the present inventors. In addition to electrically coupling the electrode 51A to the ECG wire 34 for communication of signals from the cardiac electrical activity to the electronics device 60, the removable/passthrough connector 56 allows signals from the respiratory electrical activity of the electrode R1 to be electrically coupled to the respiratory wire 40 between the electrode 51A and the electronics device 60. The removable/passthrough connector 56 is designed such that the cardiac electrical activity received at the electrode 51A remains electrically isolated from the respiratory electrical activity received at the electrode R1.

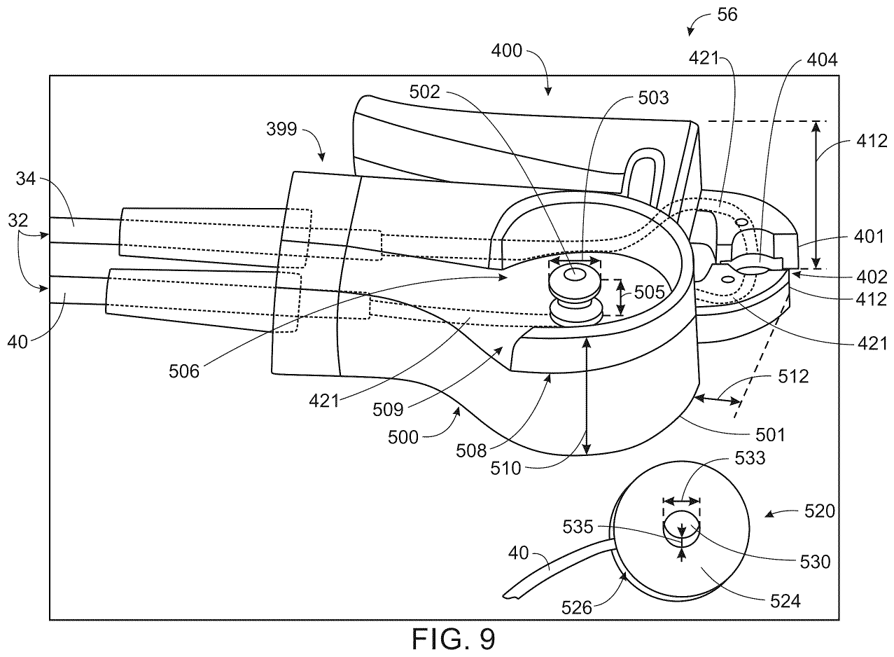

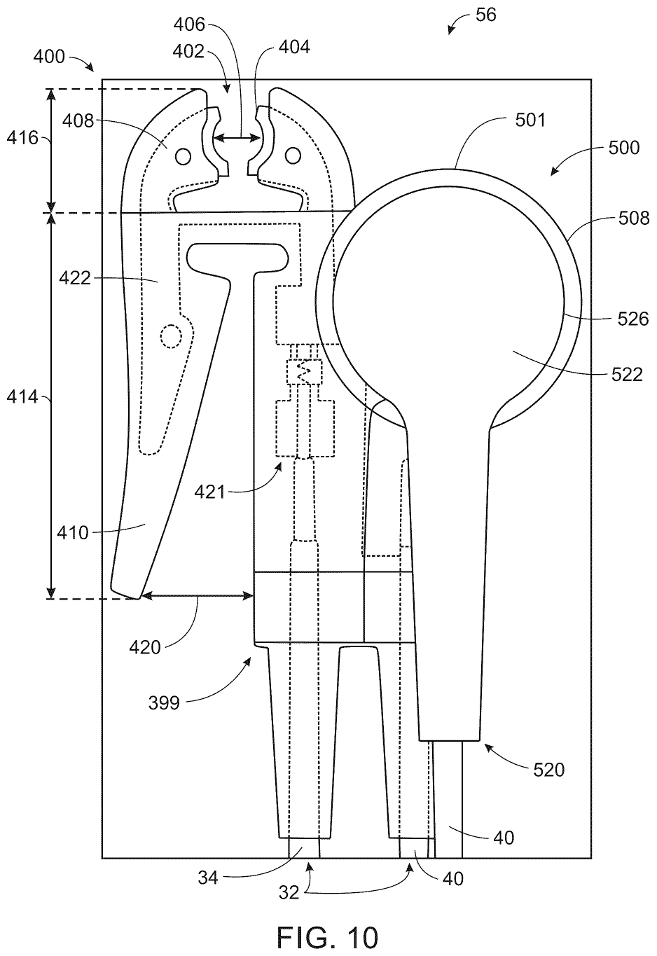

In this manner, the presently disclosed system 30 including the removable/passthrough connector 56 allows the addition of the electrode R1 simply by plugging the shared wiring harness containing both the respiratory wire 40 and the ECG wire 34 into the electronics device 60. This shared wiring harness is then connected to the electrode 51A via the removable/passthrough connector 56 (which may snap/socket or clamp on in manners known in the art), leaving the ECG wire 34 and the respiratory wire 40 electrically isolated, and also the electrodes 51A and R1 electrically isolated. It should be recognized that the electronics device 60 is also distinct from others presently known in the art, at least in that the connection for the shared wiring harness must separately receive connections for both the ECG wire 34 and the respiratory wire 40. Additional information regarding the removable/passthrough connector 56 is provided below and shown in FIGS. 9 and 10.

It should be recognized that while the above-referenced configuration is practical and cost-effective, others are also contemplated by the present disclosure. For example, the present disclosure also contemplates configurations having a separate respiratory wire 40 between the electrode R1 and the electronics device 60, rather than the shared harness and removable/passthrough connector 56 of FIG. 2.

With continued reference to the example of FIG. 2, the electrode 51B used for collecting cardiac electrical activity has a dual purpose of serving as a second electrode for respiratory data, and is thus also referred to as electrode R2. In this manner, dual vector impedance respiratory data can be collected by measuring the signals from the respiratory electrical activity between the electrodes R1 and R2, and between the electrodes R2 and R3. In certain examples, slightly different carrier frequencies are used for each of the two vector impendence measurements such that the measurements are independent of each other. For example, the frequency used for ECG data may be measured in hertz (e.g., below 150 Hz), whereas the frequency used for respiratory data may be measured in the tens of kilohertz, (e.g., between 10 and 100 kHz).

In systems and methods presently known in the art, the ground electrode is customarily placed on the right leg of the patient. Through experimentation and development, the present inventors have discovered that re-positioning the electrode G for ground (which here is also the electrode R3), specifically to the abdomen 10 of the patient 1, yields an improved signal from the respiratory electrical activity versus positioning in customary locations. For example, positioning the electrode G, R3 on the abdomen vertically approximately level to the navel 12, and near but to the left of the navel 12, provided particularly accurate readings of respiratory data.

In certain examples, it is advantageous to place the electrodes 50 where breathing efforts cause with maximum movement. For example, the upper abdominal region is generally favorable, at or above navel level. In examples in which one electrode 50, R3 is shared for both respiratory and cardiac electrical activity, it is advantageous to position the electrode 50, R3 specifically slightly to the right from navel (rather than to the left) to optimize the ECG signal amplitude.

In systems and methods presently known in the art, impedance or respiratory data measurements are measured between two ECG electrodes. Consequently, the the caregiver cannot move the shared ECG and respiratory electrode to a position to improve the quality of the incoming signal for the respiratory electrical activity. Specifically, this relocation would distort the ECG data from being positioned in a non-standard location. Accordingly, the present disclosure provides examples of systems and methods in which a ground electrode is used for measuring the respiratory data (rather than an ECG electrode), whereby this ground electrode can be placed freely without distorting ECG signals.

Additionally, the present inventors have discovered that by using a separate electrode R1 to collect the non-ground respiratory electrical activity of the patient 1 (in FIG. 2, for the first vector impedance measurement), yielded more accurate results than re-using an electrode also used for measuring ECG data. However, this is not a limitation of the presently disclosed systems and methods, and one or more of the vector impedance measurements may include an electrode 50 also used for ECG data (e.g., see electrode R2 in FIG. 2). Moreover, the ECG data and respiratory data need not share a common electrode G, R3, and need not include wires 32 that are connected directly to the electrode G, R3. For example, FIG. 2 shows only the ECG wire 34 being directly connected to the electrode G, R3, with the respiratory data obtaining this additional electrical activity via the electronics device 60 connected to both the ECG wire 34 and the respiratory wires 40, 42.

Through experimentation and development, the present inventors have further discovered a particularly advantage in positioning one of the electrodes 50 for measuring respiratory data (here, electrode R1) as shown in FIG. 2. Specifically, the present inventors have identified improvement from placing the electrode 50 horizontally closer to the left armpit 6 than to the sternum 8. In certain examples, this location is further defined as coinciding with the customary location of the V6 electrode in a 12-lead ECG (discussed further below). The present inventors have specifically noted that positioning the electrode R1 in this manner- and also as a dedicated electrode (though not required)—yields a strong, accurate signal representing the respiratory electrical activity of the patient 1.

FIG. 2 also shows that the system 30 is configured to be portable, having an electronics device 60 that can move with the patient 1. In the example shown, the electronics device 60 is retained on the patient via a belt 70 (e.g., by a clip, hook and loop fastener, or other methods known in the art). This allows the patient 1 to move about while the system 30 collects the ECG and/or respiratory data, which is both convenient, and in some cases necessary for testing protocols (e.g., a cardiac stress test). Additional flexibility is provided when the connection 28 between the electronics device 60 and the external monitoring device 20 (see FIG. 1) is wireless.

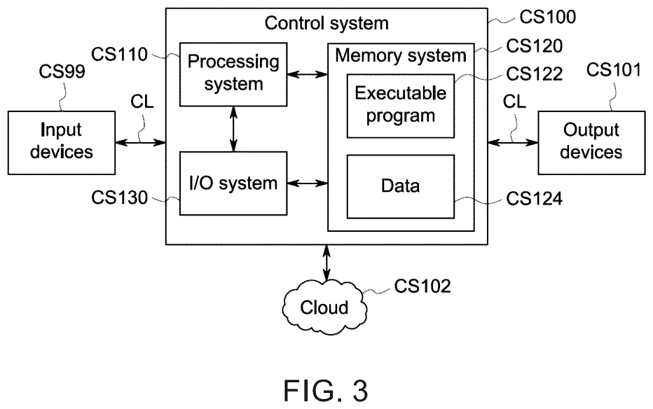

The electronics device 60 of FIG. 2 may be or may incorporate a control system CS100 such as shown in FIG. 3, whereby the wires 32 constitute the input devices CS99 thereto and the monitoring device 20 (FIG. 1) constitutes an example of output device CS101. The control system CS100 receives and processes the electrical signals received from the wires, which may be passed to an output device CS101, and/or processed via a processing system CS110 to generate the ECG data for the patient (e.g., as a waveform displayed on a display device).

It should be recognized that the electronics device 60 and the monitoring device 20 may be incorporated into a single device, or subdivided from the examples discussed herein while preserving the same function. Likewise, there may be multiple control systems configured like the control system CS100 of FIG. 3, for example in each electronics device 60 and the monitoring device 20. In certain examples, the control system CS100 of the electronics devices 60 merely communicate the electrical activity received from the electrodes 50 to the monitoring device 20, whereby a control system CS100 thereon processes this electrical activity to generate the ECG data, ECG waveforms, notifications, and the like.

As stated above, FIG. 3 depicts an example of a control system CS100 such as may be incorporated within the system 30, here specifically within the electronics device 60. Certain aspects of the present disclosure are described or depicted as functional and/or logical block components or processing steps, which may be performed by any number of hardware, software, and/or firmware components configured to perform the specified functions. For example, certain embodiments employ integrated circuit components, such as memory elements, digital signal processing elements, logic elements, look-up tables, or the like, configured to carry out a variety of functions under the control of one or more processors or other control devices. The connections between functional and logical block components are merely examples, which may be direct or indirect, and may follow alternate pathways.

In certain examples, the control system CS100 communicates with each of the one or more components of the system 30 via a communication link CL (e.g., wires 32 and connections 28 in FIGS. 1 and 2), which can be any wired or wireless link. The control module CS100 is capable of receiving information and/or controlling one or more operational characteristics of the system 30 and its various sub-systems by sending and receiving control signals via the communication links CL. In one example, the communication link CL is a controller area network (CAN) bus; however, other types of links could be used. It will be recognized that the extent of connections and the communication links CL may in fact be one or more shared connections, or links, among some or all of the components in the system 30. Moreover, the communication link CL lines are meant only to demonstrate that the various control elements are capable of communicating with one another, and do not represent actual wiring connections between the various elements, nor do they represent the only paths of communication between the elements. Additionally, the system 30 may incorporate various types of communication devices and systems, and thus the illustrated communication links CL may in fact represent various different types of wireless and/or wired data communication systems.

The control system CS100 may be a computing system that includes a processing system CS110, memory system CS120, and input/output (I/O) system CS130 for communicating with other devices, such as input devices CS99 and output devices CS101 (e.g., a monitoring device 20, an Electronic Medical Record, and/or other external devices (e.g., smart phones or tablets), which may also or alternatively be stored in a cloud 102. The processing system CS110 loads and executes an executable program CS122 from the memory system CS120, accesses data CS124 stored within the memory system CS120, and directs the system 30 to operate as described in the present disclosure.

The processing system CS110 may be implemented as a single microprocessor or other circuitry, or be distributed across multiple processing devices or sub-systems that cooperate to execute the executable program CS122 from the memory system CS120. Non-limiting examples of the processing system include general purpose central processing units, application specific processors, and logic devices.

The memory system CS120 may comprise any storage media readable by the processing system CS110 and capable of storing the executable program CS122 and/or data CS124. The memory system CS120 may be implemented as a single storage device, or be distributed across multiple storage devices or sub-systems that cooperate to store computer readable instructions, data structures, program modules, or other data. The memory system CS120 may include volatile and/or non-volatile systems, and may include removable and/or non-removable media implemented in any method or technology for storage of information. The storage media may include non-transitory and/or transitory storage media, including random access memory, read only memory, magnetic discs, optical discs, flash memory, virtual memory, and non-virtual memory, magnetic storage devices, or any other medium which can be used to store information and be accessed by an instruction execution system, for example.

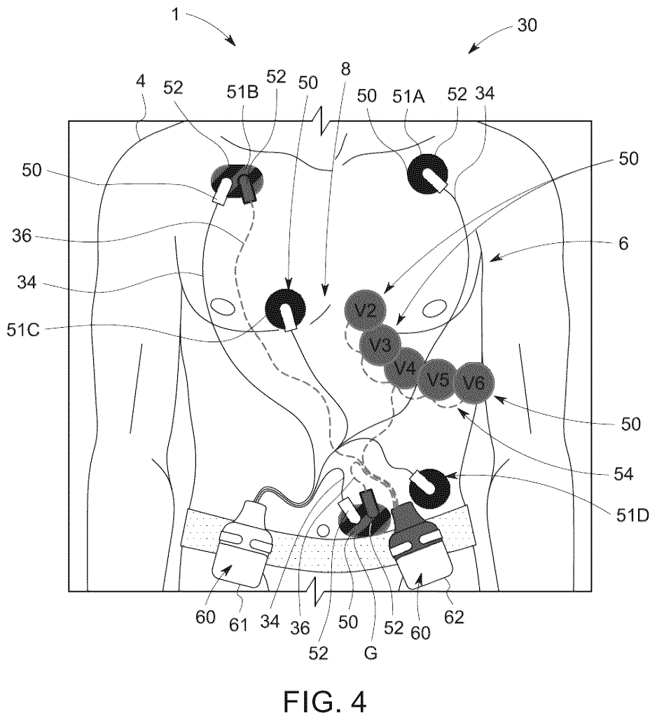

FIG. 4 shows another configuration for a system 30 configured to measure ECG data, this time not showing electrodes for measuring respiratory data. The system 30 includes the same ECG wires 34 connected to a first electronics device 61 as shown in FIG. 2, which is also referred to as a first set of ECG wires communicating a first set of cardiac electrical activity. In the configuration of FIG. 4, a second set of ECG wires communicating a second set of cardiac electrical activity has been added to the first set. Specifically, this includes additional electrodes 50 and additional ECG wires 36 connected to a second electronics device 61 as the second set of ECG wires communicating the second set of cardiac electrical activity. The electrodes 50, ECG wires 34, 46, and first and second electronics devices 61, 62 may be functionally the same between the first and second sets unless otherwise noted.

By adding the second set of ECG wires 36 to the first set of ECG wires 34 from FIG. 2, the system 30 is expanded from a 5-lead ECG configuration to a full, 12-lead ECG setup. This allows the caregiver to conduct the more extensive analysis and testing of a full 12-lead ECG, without requiring the removal of the electrodes 50 already in position from the previous 5-lead ECG monitoring associated with the first set of ECG wires. By utilizing the existing electrodes of the 5-lead ECG in the 12-lead ECG, time and effort is saved, the cost of materials is reduced, the patient remains more comfortable, and human error is reduced, as discussed above.

In the example shown in FIG. 4, the first and second sets of ECG wires 34, 36 have a shared or common ground electrode G, which in this case has two removable connector 52 (e.g., clamps or snaps). However, it should be recognized that separate ground electrodes may be used.

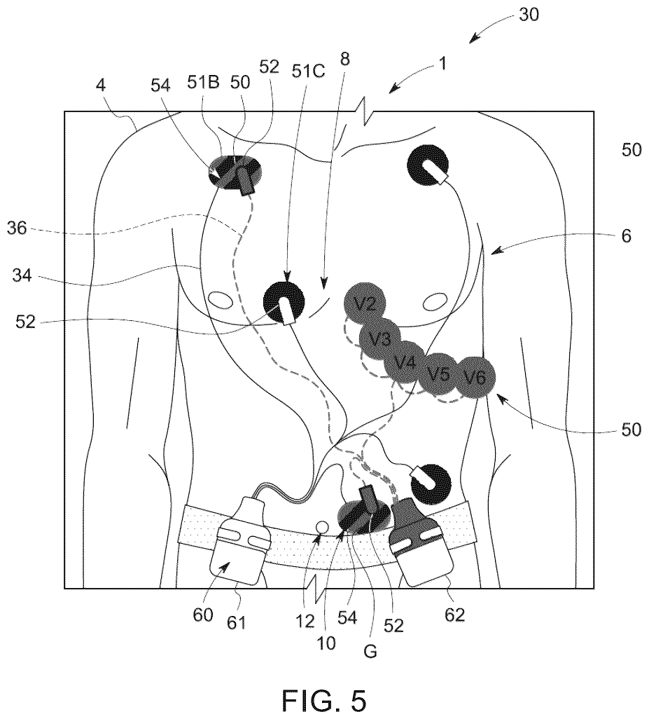

A similar configuration having the same placement of electrodes 50 is shown in FIG. 5. The system 30 of FIG. 5 includes electrodes 50 having three different types of connectors for connecting wires 32 thereto. In particular, some electrodes (e.g., electrode 51C) are connectable to a single wire 32 via removable connector 52 (e.g., a snap or clamp as known in the art). Other electrodes 50 have fixed connectors 54, meaning they are hard-wired or permanently coupled to the wire 32 (e.g., electrodes V2-V6), and still further electrodes 50 have both a fixed connector 54 and a removable connector 52 (e.g., electrode 51B). The present inventors have recognized that utilizing fixed connectors 54 allows at least some of the wires 32 within the 12-lead ECG to be made as a simplified and disposable assembly (e.g., the electrodes V2-V6 being connected as a single, fixed unit, ensuring proper placement therebetween), whereby electrodes 50 having both a fixed connector 54 and a removable connector 52 allows the user to subsequently add on to the already placed electrode, such as electrode 51B. Configuring electrode 51B to be a fixed connector 54 for the first set of cardiac electrical activity, while providing the removable connector 52, allows the same electrode 51B to later be used for a second set of cardiac electrical activity as needed (thereby reducing time, cost, and patient discomfort). It should be recognized that the particular configuration of fixed connectors 54 and removable connectors 52 may vary from that shown.

The ECG data received at the first and second electronics devices 61, 62 may be combined together (e.g., within either one of the electronics devices 60, for example via a wired or wireless connection therebetween), and/or may be passed independently to output devices (CS101, FIG. 3) for combination thereon. For example, a monitoring device (20 of FIG. 1) may be configured to select between 5-lead and 12-lead configurations, receiving, processing, and/or displaying the corresponding ECG data on the display device 22 accordingly. This selection may also be made by the monitoring device 20 automatically based on whether or not it is communicating with one or two electronic devices 60, for example. In certain examples, the system 30 may be configured to generate and transmit an alarm or notification on the display device 22 or a third party devices (e.g., a text message or other communication to a third party device, such as a caregiver smart phone) when one of the electronic devices 60 is connected to the patient 1 and receiving electrical activity therefrom, but the monitoring device 20 is configured such that that electrical activity is not being stored, used, and/or displayed, for example. The same alarms or notifications may also be provided when the monitoring device 20 is in a mode (e.g., 12-lead ECG mode), but not receiving electrical activity from all necessary electronic devices 60. Specific details regarding which of the electronics devices 60 is not communicating with the monitoring device 20, and/or any wires between the electronics devices 60 and the electrodes 50 may also be included in the alarms and notifications to aid in troubleshoot or reconfiguring the system 30.

The monitoring device 20 may be part of the system 30 itself, and/or may contain a control system CS100 such as that shown in FIG. 3 for receiving, processing, displaying, and performing other functions using the ECG data measured by the electronic devices 60 (whether one or two electronics devices). It should be recognized that in this example, the monitoring device 20 may be different than those presently known in the art, particularly to provide the connectivity and processing of information coming from the electronic devices 60 presently disclosed.

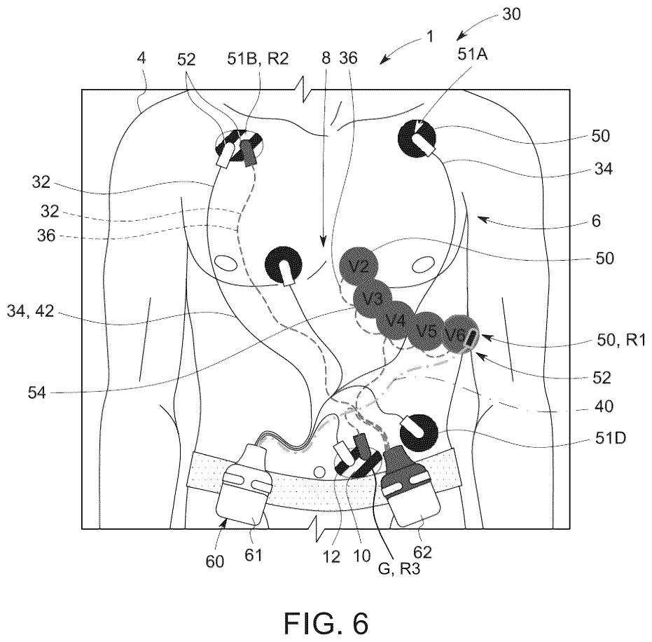

FIG. 6 shows another system 30 similar to that shown in FIG. 4, but now also configured to measure respiratory data. In the example shown, electrode V6 used for measuring ECG data (here, connected as a fixed connector 54 to a wire 36 within the second set of ECG wires 36 to the second electronics device 62) also includes a removable connector 52 for connecting a respiratory wire 40. In this manner, electrode V6 also serves as electrode R1, being positioned near the left armpit 6 as identified by the present inventors to be particularly advantageously. The electrode 51B used for both the first and second sets of ECG wires 32, 34 is also used as the respiratory electrode for the second vector impedance and is thus also labeled as electrode R2. In this example, a separate respiratory wire 40 is not provided, instead obtaining this respiratory electrical activity from the wire 32 already connected to the first electronics device 61.

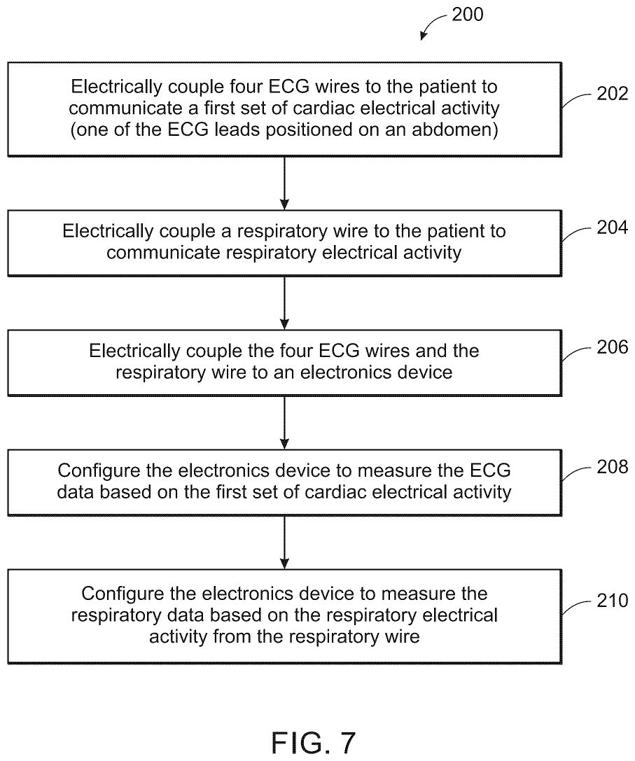

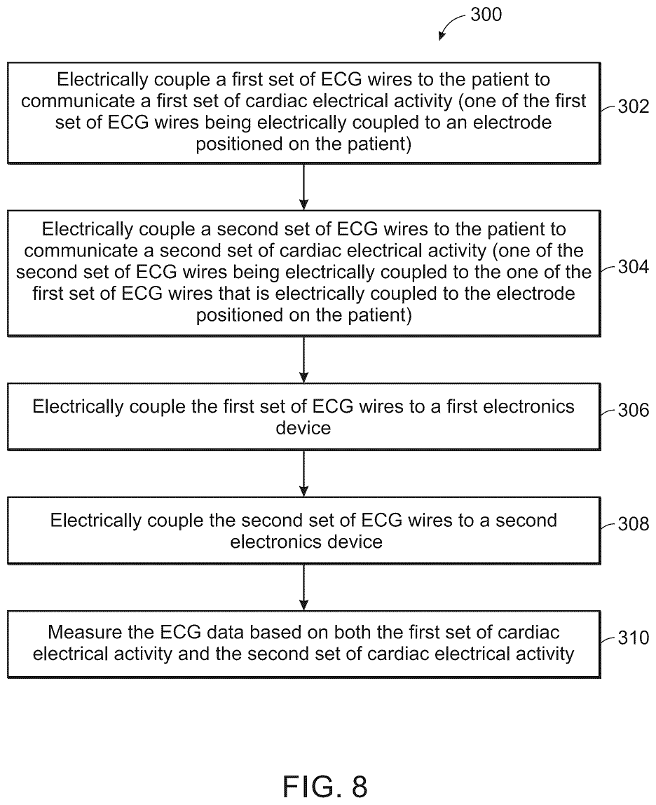

FIGS. 7 and 8 are flow charts of example methods 200 and 300 for measuring ECG data according to the present disclosure, respectively, for example using one of the systems 30 described above. While the present flow charts reflect a 4-lead ECG setup, other numbers of leads are also contemplated by the present disclosure. In particular, FIG. 7 provides for electrically coupling (in step 202) four (or more) ECG wires to the patient to communicate a first set of cardiac electrical activity (one of the ECG leads positioned on an abdomen). Step 204 provides for electrically coupling a respiratory wire to the patient to communicate respiratory electrical activity. Step 206 provides for electrically coupling the four (or more) ECG wires and the respiratory wire to an electronics device. Steps 208 and 210 provide for configuring the electronics device to measure the ECG data based on the first set of cardiac electrical activity, and configuring the electronics device to measure the respiratory data based on the respiratory electrical activity from the respiratory wire.

In the method 300 of FIG. 8, step 302 provides for electrically coupling a first set of ECG wires to the patient to communicate a first set of cardiac electrical activity (one of the first set of ECG wires being electrically coupled to an electrode positioned on the patient). Step 304 provides for electrically coupling a second set of ECG wires to the patient to communicate a second set of cardiac electrical activity (one of the second set of ECG wires being electrically coupled to the one of the first set of ECG wires that is electrically coupled to the electrode positioned on the patient). Steps 306 and 308 include electrically coupling the first set of ECG wires to a first electronics device, and electrically coupling the second set of ECG wires to a second electronics device. In step 310, the ECG data is measured based on both the first set of cardiac electrical activity and the second set of cardiac electrical activity.

FIGS. 9 and 10 show an example of a removable/passthrough connector 56 according to the present disclosure, which as described may be used to enable systems 30 according to the present disclosure to be easily expanded with the addition of a second electronics device 60 and associated electrodes 50 as needed. The removable/passthrough connector 56, and/or the removable portion 52 connectable thereto, may be reusable or disposable depending on the application. Likewise, the removable/passthrough connector 56 is not limited to use with the systems 30 and methods described herein, not to ECG contexts. Other exemplary uses include electromyography (EMG), electroencephalography (EEG), or any other systems or devices in which wires are connected to contacts (by way of non-limiting example, electrodes). In the example shown, the removable/passthrough connector 56 comprises a first connector 400 and a second connector 500 that share a joint body 399. The first connector 400 extends to a first end 401 having a clamp 402 designed for clamping to an electrode positioned on the skin of the patient in a customary manner. Specifically, the clamp 402 includes contacts 404 supported by support arms 408 and separated by an opening 406. The opening 406 may be temporarily increased, for example to remove the first connector 400 from an electrode, by pressing pinch arms 410 together in the customary manner, thereby reducing a gap 420 therebetween.

The joint body 399, and particularly within the first connector 400, is resilient such that when the pinch arms 410 are not pressed together, the opening 406 between the clamps 402 corresponds to the size and shape of the electrode to be clamped onto. The lengths 414, 416 of the pinch arms 410 and the support arms 408, respectively, are designed to provide the necessary leverage for an operator to easily open the clamp 402 when desired, which is also a function of the resiliency of the materials selected. It should be recognized that the clamp 402 may be biased in the closed position shown in FIGS. 9 and 10 by other methods known in the art, including through the use of springs.

In the example shown, the height 412 of the first connector 400 also varies, here being less at the clamp 402 than at the pinch arms 410. This provides for additional surface area where the user presses the pinch arms 410 together, but also a low enough provide to engage a customary electrode. Likewise, the first end 401 of the first connector 400 may be offset forward from the first end 501 of the second connector 500 by an offset 512. This ensures that the first end 501 of the second connector 500 does not interfere with the connection and disconnection of the first connector.

With continued reference to FIGS. 9 and 10, the joint body 399 further includes the second connector 500, which is electrically isolated from the first connector 400 as discussed above. The second connector 500 extends from a first end 501 and includes a contact 502 for electrically engaging with a removable portion 520 when connected thereto. In the example shown, the contact 502 is a male-end nipple, which may be the same or similar to the male contact of an electrode presently known in the art (including that which the clamp 402 of the first connector 400 is configured to engage). The contact 502 extends upwardly by a height from a floor 506 on which the removable portion 520 rests when connected to the second connector 500. Walls 508 also extend upwardly from the floor 506 having a height 510 from the bottom of the second connector 500.

The walls 508 are sized and shaped to correspond to the sides 526 of the removable portion 520 such that the removable portion 520 is secure therein and prevented from accidental removal (e.g., shear forces from catching on other wires, equipment, and the like). The walls 508 also provide increased electrical safety for the patient, effectively shielding the contact 502 from accidental contact with other electrical devices. Likewise, the walls 508 serve as a mistake-proofing mechanism to ensure that only the intended removable portion 520 is connected to the second connection 500 (via the corresponding shapes and sizes thereof).

The walls 508 also provide for cable management of the wires 32 for the removable/passthrough connector 56. In particular, a gap 509 is formed between the walls 508, in this example generally opposite the first end 501 of the second connector 500. The gap 509 is the only opening through which the respiratory wires 40 (or other wires in other contexts) may extend when the removable portion 520 is engaged within the second connector 500. In this example, this alignment via the gap 509 causes the respiratory wire 40 connected to the removable portion 52 to be aligned in parallel to the wires 32 embedded within the joint body 399. It should be recognized that these wires 32 are electrically coupled to the contacts 404, 502 of the first connector 400 and the second connector 500, respectively, via internal wires 421. The internal wires 421 may be integrally formed within the joint body 399 as an overmold in a manner known in the art, for example. In certain examples (e.g., FIG. 9), internal wires 421 may run internally to connect the wires 32 with the contacts 404 and/or 502. In other examples (e.g., FIG. 10), the wires 32 may be connected to an internal wire 421 that is in turn connected to the contacts 404, 502 via a conductive plate 422, for example. In the example shown, the conductive plate 422 forms the contacts 404 of the first connector 400.

With continued reference to FIGS. 9 and 10, the walls 508 of the removable portion 520 extend between an outside 522 and an inside 524, here forming a generally cylindrical shape. As shown in FIG. 9, a second contact 530 is provided on or within the inside 524 of the removable portion 520. In this example, the second contact 530 is generally circular and has a diameter 533 and depth 535 corresponding to the diameter 503 and height 505 of the first contact 502 such that a snap-type connection is formed therebetween, for example as used with snap-type electrode connections in the art. It should be recognized that the actual conductive portion of the second contact 530 may not mirror the complete cylindrical shape of the opening defined by the diameter 533 and depth 535 defined within the removable portion 520. In this manner, the removable portion 520 is electrically coupled to the removable/passthrough connection by forcing the inside 524 against the floor 506 of the joint body 399. Likewise, the removable portion 520 may be removed (e.g., when no longer needed), but pulling the removable portion 520 in a direction normal to the floor 506.

It should be recognized that the contacts 530, 502 of the removable portion 520 and the second connector 500 within the joint body 399 may be reversed, and/or other types of connections may be substituted to provide the similar functionality. The present inventors have noticed multiple benefits of using removable/passthrough connectors 56, including but not limited to use within the systems 30 described above. In particular, the removable/passthrough connectors 56 described above are unobtrusive and provide for fast and easy connection and disconnection of the removable portion 520 as needed. Each of the first connector 400 and second connector 500 are also very intuitive to caregivers, requiring no special training and allowing instant identification of whether either connector is properly connected.

In this manner, the systems and methods disclosed herein provide for an improved workflow, improved flexibility, and improved accuracy of measuring ECG and respiratory data in patients. Furthermore, less equipment is needed at a care facility as there is no longer a need to have both 5-lead ECG devices for long-term monitoring versus 12-lead ECG devices for short-term testing, for example.

The functional block diagrams, operational sequences, and flow diagrams provided in the Figures are representative of example architectures, environments, and methodologies for performing novel aspects of the disclosure. While, for purposes of simplicity of explanation, the methodologies included herein may be in the form of a functional diagram, operational sequence, or flow diagram, and may be described as a series of acts, it is to be understood and appreciated that the methodologies are not limited by the order of acts, as some acts may, in accordance therewith, occur in a different order and/or concurrently with other acts from that shown and described herein. For example, those skilled in the art will understand and appreciate that a methodology can alternatively be represented as a series of interrelated states or events, such as in a state diagram. Moreover, not all acts illustrated in a methodology may be required for a novel implementation.

This written description uses examples to disclose the invention, including the best mode, and also to enable any person skilled in the art to make and use the invention. Certain terms have been used for brevity, clarity, and understanding. No unnecessary limitations are to be inferred therefrom beyond the requirement of the prior art because such terms are used for descriptive purposes only and are intended to be broadly construed. The patentable scope of the invention is defined by the claims and may include other examples that occur to those skilled in the art. Such other examples are intended to be within the scope of the claims if they have features or structural elements that do not differ from the literal language of the claims, or if they include equivalent features or structural elements with insubstantial differences from the literal languages of the claims.

What is claimed is:

1. A method for monitoring a patient, the method comprising:
connecting a first set of ECG wires to the patient so as to receive a first set of cardiac electrical activity from the patient therewith;
connecting a second set of ECG wires to the patient so as to receive a second set of cardiac electrical activity from the patient therewith;
monitoring the patient based on the first set of cardiac electrical activity and based on the second set of cardiac activity when the first set of ECG wires and the second set of ECG wires are connected to the patient;
disconnecting the second set of ECG wires from the patient without disconnecting the first set of ECG wires from the patient; and
monitoring the patient based on the first set of cardiac electrical activity when the second set of ECG wires is disconnected from the patient;
wherein optionally connecting the second set of ECG wires allows the patient to be monitored in two different configurations without disconnecting the first set of ECG wires from the patient to transition therebetween.

2. The method according to claim 1, wherein the patient is monitored in a 5-lead or 6-lead ECG configuration when the second set of ECG wires is disconnected from the patient and in a 12-lead configuration when the second set of ECG wires is connected to the patient.

3. The method according to claim 1, wherein the first set of ECG wires and the second set of ECG wires are coupled to the patient via electrodes, and wherein the electrodes connected to the first set of ECG wires are left in position on the patient in the two different configurations.

4. The method according to claim 3, further comprising connecting at least one of the first set of ECG wires and at least one of the second set of ECG wires to the patient via a shared electrode within the electrodes positioned on the patient.

5. The method according to claim 1, further comprising electrically coupling the first set of ECG wires to a first control system and electrically coupling the second set of ECG wires to a second control system, and wherein the second control system is separate from the first control system such that the second control system can be disconnected when the second set of ECG wires is disconnected from the patient.

6. The method according to claim 5, further comprising securing the first control system to the patient so as to be movable therewith.

7. The method according to claim 5, further comprising electrically coupling the first control system to a monitoring device configured to measuring ECG data for monitoring the patient based on the first set of cardiac electrical activity, wherein the first control system is physically separatable from the monitoring device.

8. The method according to claim 1, further comprising electrically coupling the first set of ECG wires to a first control system and electrically coupling the first control system to a monitoring device configured to measuring ECG data for monitoring the patient based on the first set of cardiac electrical activity, wherein the first control system is physically separatable from the monitoring device so as to be movable with the patient.

9. A method for measuring electrocardiogram (ECG) data for a patient, the method comprising:
connecting a first set of ECG wires to the patient so as to receive a first set of cardiac electrical activity from the patient therewith;
electrically coupling the first set of ECG wires to a monitoring device;
connecting a second set of ECG wires to the patient so as to receive a second set of cardiac electrical activity from the patient therewith;
electrically coupling the second set of ECG wires to the monitoring device;
measuring the ECG data for the patient based on the first set of cardiac electrical activity and based on the second set of cardiac electrical activity when both the first set of ECG wires and the second set of ECG wires are electrically coupled to the monitoring device, and measuring the ECG data for the patient based on the first set of cardiac electrical activity without being based on the second set of cardiac electrical activity when the second set of ECG wires is uncoupled from the monitoring device.

10. The method according to claim 9, wherein the step of measuring the ECG data based on the first set of cardiac electrical activity without being based on the second set of cardiac electrical activity is performed before electrically coupling the second set of ECG wires.

11. A system for measuring electrocardiogram (ECG) data for a patient, the system comprising:
a first control system configured to be electrically coupled to the patient via a first set of ECG wires to receive a first set of cardiac electrical activity from the patient;
a second control system configured to be electrically coupled to the patient via a second set of ECG wires to receive a second set of cardiac electrical activity from the patient; and
a monitoring device configured to communicate with the first control system and the second control system, wherein the monitoring device is configured to measure the ECG data for the patient based on the first set of cardiac electrical activity received from the first control system when communication is absent from the second control system, and wherein the monitoring device is configured to measure ECG data for the patient based on both the first set of cardiac electrical activity received from the first control system and the second set of cardiac electrical activity received from the second control system when communication is present from both the first control system and the second control system.

12. The system according to claim 11, wherein the system operates as a 5-lead ECG configuration or a 6-lead ECG configuration when communication is absent from the second control system, and as a 12-lead ECG configuration when communication is present from both the first control system and the second control system.

13. The system according to claim 12, wherein the system is configured to transition between operating as the 5-lead ECG configuration or the 6-lead ECG configuration and operating as the 12-lead ECG configuration without electrically disconnecting the first control system from the monitoring device.

14. The system according to claim 12, wherein the system is configured to transition between operating as the 5-lead ECG configuration or the 6-lead ECG configuration and operating as the 12-lead ECG configuration without disconnecting the first set of ECG wires from the patient.

15. The system according to claim 11, wherein the first set of ECG wires is coupled to the monitoring device through the first control system and the second set of ECG wires is coupled to the monitoring device through the second control system.

16. The system according to claim 11, wherein the second set of ECG wires comprise leads V2 through V6 in a conventional 12-lead ECG configuration.

17. The system according to claim 11, wherein the monitoring device is configured to measure ECG data for the patient based on both the first set of cardiac electrical activity and the second set of cardiac electrical activity when at least one of the first set of ECG wires and at least one of the second set of ECG wires are electrically coupled to the patient via a shared electrode positioned thereon.

18. The system according to claim 17, wherein the shared electrode provides additional electrical activity for both the first set of ECG wires and the second set of ECG wires, and wherein measuring the ECG data includes comparing each of the first set of cardiac electrical activity and the second set of cardiac electrical activity to the additional electrical activity.

19. The system according to claim 11, wherein the first control system is further configured to be electrically coupled to the patient via a respiratory wire configured to measure respiratory electrical activity for the patient, wherein the respiratory wire is distinct from the first set of ECG wires, and wherein the monitoring device is further configured to measure respiratory data for the patient based on the respiratory electrical activity received from the respiratory wire.

20. A method for using the system of claim 11, the method comprising electrically coupling the first set of ECG wires to the patient via electrodes, wherein one of the electrodes is positioned on an abdomen of the patient.
